(19) United States
(12) Patent Application Publication
(10) Pub. No.: US 2016/0143924 A1
(43) Pub. Date: May 26, 2016

(54) LOW DOSE PHARMACEUTICAL COMPOSITION OF DOXYCYCLINE

(71) Applicant: SUN PHARMACEUTICAL INDUSTRIES LIMITED, Mumbai (IN)

(72) Inventors: Dileep JAMI, Srikakulam (IN); Ravinder SINGH, Gurgaon (IN); Anuj Kumar FANDA, Ghaziabad (IN); Romi Barat SINGH, Varanasi (IN)

(21) Appl. No.: 14/821,280

(22) Filed: Aug. 7, 2015

Related U.S. Application Data
(63) Continuation-in-part of application No. 14/548,915, filed on Nov. 20, 2014.

(51) Int. Cl. A61K 31/65

(52) U.S. Cl. CPC A61K 31/65

(57) ABSTRACT

The present invention relates to a low dose pharmaceutical composition comprising 24 mg to 36 mg doxycycline and one or more pharmaceutically acceptable excipients for the treatment of acne and/or rosacea, and processes for its preparation.

### FIELD OF THE INVENTION

The present invention relates to a low dose pharmaceutical composition of doxycycline comprising 24 mg to 36 mg of doxycycline and one or more pharmaceutically acceptable excipients for the treatment of acne and/or rosacea, and processes for its preparation.

### BACKGROUND OF THE INVENTION

U.S. Pat. No. 8,652,516 discloses a pharmaceutical composition of doxycycline comprising a capsule, wherein the capsule is coated with a delayed-release layer which comprises about 4 mg to 6 mg of doxycycline and an immediate-release layer which comprises about 32 mg of doxycycline. This patent discloses 36 mg and 38 mg doxycycline capsule formulations which are bioequivalent to the reference product Oracea® 40 mg.

Doxycycline has gastrointestinal side effects which are dose related, such as gastrointestinal irritation. Thus, lowering the dose of doxycycline may reduce these side effects. The present inventors have prepared a pharmaceutical composition which further lowers the dose of doxycycline for the treatment of acne and/or rosacea.

The present invention is directed to a low dose pharmaceutical composition comprising 24 mg to 36 mg doxycycline which is effective in the treatment of acne and/or rosacea.

### SUMMARY OF THE INVENTION

The present invention provides a low dose pharmaceutical composition of doxycycline comprising 24 mg to 36 mg of doxycycline and one or more pharmaceutically acceptable excipients for the treatment of acne and/or rosacea, and processes for its preparation.

### DETAILED DESCRIPTION OF THE INVENTION

A first aspect of the present invention provides a low dose pharmaceutical composition comprising 24 mg to 36 mg of doxycycline and one or more pharmaceutically acceptable excipients.

According to one embodiment of this aspect, the low dose pharmaceutical composition is intended for once daily administration.

According to another embodiment of the above aspect, the low dose pharmaceutical composition comprises 30 mg to 36 mg of doxycycline.

According to another embodiment of the above aspect, the low dose pharmaceutical composition comprises 32 mg to 36 mg of doxycycline.

According to another embodiment of the above aspect, the low dose pharmaceutical composition comprises 24 mg of doxycycline.

According to another embodiment of the above aspect, the low dose pharmaceutical composition comprises 26 mg of doxycycline.

According to another embodiment of the above aspect, the low dose pharmaceutical composition comprises 28 mg of doxycycline.

According to another embodiment of the above aspect, the low dose pharmaceutical composition comprises 30 mg of doxycycline.

According to another embodiment of the above aspect, the low dose pharmaceutical composition comprises 32 mg of doxycycline.

According to another embodiment of the above aspect, the low dose pharmaceutical composition comprises 34 mg of doxycycline.

According to another embodiment of the above aspect, the low dose pharmaceutical composition comprises 36 mg of doxycycline.

According to another embodiment of this aspect, the low dose pharmaceutical composition is an immediate release composition.

A second aspect of the present invention provides a method of treating acne and/or rosacea by administering a low dose pharmaceutical composition comprising 24 mg to 36 mg of doxycycline and one or more pharmaceutically acceptable excipients.

The pharmaceutical composition may be in the form of capsules or tablets. Preferably, the pharmaceutical composition is in the form of tablets.

The term “doxycycline,” as used herein, includes doxycycline base and its pharmaceutically acceptable salts, hydrates, solvates, esters, or prodrugs. Preferably, doxycycline is used as its hyclate salt, which is doxycycline hydrochloride hemiethanolate hemihydrate.

The term “low dose,” as used herein, refers to a dose of 24 mg to 36 mg doxycycline base, which is less than the conventional dose of 40 mg which is required to produce the therapeutic effect.

The term “pharmaceutically acceptable excipients,” as used herein, includes any physiologically inert additives that are routinely used in pharmaceutical compositions. Pharmaceutically acceptable excipients are selected from the group comprising binders, diluents, disintegrants, lubricants/glidants/antiadherants, acidifying agents and mixtures thereof. The pharmaceutically acceptable excipients may be added intragranularly as well as extragranularly.

Examples of binders include povidone, copovidone, polyvinyl pyrrolidone, hydroxypropylmethyl cellulose, hydroxypropyl cellulose, hydroxyethyl cellulose, methyl cellulose, ethyl cellulose, xanthan gum, gum acacia, gum arabic, tragacanth, sorbitol, dextrose, sucrose, mannitol, gelatin, pullulan, sodium alginate, calcium alginate, ammonium calcium alginate, propylene glycol, polyvinyl alcohol, corn syrup, methacrylates, carboxyvinyl polymers like carbomers, and mixtures thereof.

Examples of diluents include microcrystalline cellulose, powdered cellulose, dibasic calcium phosphate, tribasic calcium phosphate, calcium sulfate, calcium carbonate, lactose monohydrate, lactose anhydrous, sucrose, sorbitol, xylitol, erythritol, kaolin, calcium silicate, maltodextrin, starch, modified starch, e.g., pregelatinized starch, maize starch, corn starch, and mixtures thereof.

Examples of disintegrants include hydroxypropyl cellulose (L-HPC), crospovidone, croscarmellose sodium, carboxymethyl cellulose sodium, carboxymethyl cellulose calcium, sodium starch glycolate, gums, alginic acid or alginates, starch, corn starch, modified starch, carboxymethyl starch, polyacrylates, and mixtures thereof.

Examples of lubricants/glidants/antiadherents include magnesium stearate, hydrogenated vegetable oil, glyceryl behenate, glyceryl monostearate, stearic acid, sodium stearyl fumarate, calcium stearate, zinc stearate, aluminum silicate, talc, colloidal silicon dioxide, sucrose esters of fatty acids, waxes, silica gel, and mixtures thereof.

Acidifying agents are pH modifiers which provide an acidic environment required for stability of the drug. Examples of acidifying agents include citric acid, tartaric acid, adipic acid, fumaric acid, malic acid, acetic acid, lactic acid, hydrochloric acid, phosphoric acid, and mixtures thereof.

Various solvents that may be employed during the preparation of the pharmaceutical composition of the present invention are selected from the group comprising methyl alcohol, ethyl alcohol, isopropyl alcohol, n-butyl alcohol, acetone, acetonitrile, chloroform, methylene chloride, water, and mixtures thereof.

The pharmaceutical composition of the present invention may be prepared by any of the well-known processes including wet granulation, dry granulation, direct compression, top spray granulation, and drug layering.

The pharmaceutical composition of the present invention may be in the form of a tablet or a capsule. The tablet or capsule may be further coated with a film coating prepared by using a film-forming polymer and one or more pharmaceutically acceptable excipients. The pharmaceutically acceptable excipients may be plasticizers, opacifiers, coloring agents, and mixtures thereof.

Examples of film-forming polymers include hydroxypropylmethyl cellulose, ethyl cellulose, methyl cellulose, hydroxyethyl cellulose, hydroxypropyl cellulose, sodium carboxymethyl cellulose, cellulose acetate, hydroxypropylmethyl cellulose phthalate, cellulose acetate trimellitate, methacrylic acid copolymers, e.g., Eudragit®, polyvinylpyrrolidone, polyvinylalcohol, polyethylene glycol, and mixtures thereof. A preferred film-forming polymer is hydroxypropylmethyl cellulose. Other suitable film-forming polymers which are known in the art may also be used. Many suitable film coating products which are commercially available, such as Opadry® and Opaglos®, may be used.

Examples of plasticizers include propylene glycol, triethyl citrate, tributyl citrate, dibutyl sebacate, acetyl tributyl citrate, glyceryl monostearate, triacetin, polyethylene glycol, diethyl phthalate, acetylated monoglycerides, diacetylated monoglycerides, cetyl alcohol, and mixtures thereof.

Examples of opacifiers include titanium dioxide, manganese dioxide, iron oxide, silicon dioxide, and mixtures thereof.

The coloring agents may be selected from FDA approved colorants such as iron oxide, lake of tartrazine, allura red, titanium dioxide, and mixtures thereof.

The coating may be carried out by using any conventional coating techniques known in the art, such as spray coating in a conventional coating pan or fluidized bed processor, or dip coating.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

The following examples illustrate the present invention but are not to be construed as limiting the scope of the invention.

### EXAMPLES

### Example 1

| | Quantity |
|---|---|
| Ingredients | (% w/w) |
| Doxycycline hyclate equivalent to doxycycline 32 mg | 18.47 |
| Hydroxypropylmethyl cellulose | 1.54 |
| Sugar spheres | 80.00 |
| Purified water | q.s. |

### Manufacturing Process:

1. Hydroxypropylmethyl cellulose is dissolved in purified water to form a binder solution.
2. Doxycycline is added to the binder solution of step 1 to form a drug-binder solution.
3. Sugar spheres are coated with the drug-binder solution of step 2 to obtain coated pellets.
4. The coated pellets of step 3 are dried, then filled into capsules.

### Example 2

| | Quantity |
|---|---|
| Ingredients | (% w/w) |
| Doxycycline hyclate equivalent to doxycycline 34 mg | 19.62 |
| Hydroxypropylmethyl cellulose | 2.38 |
| Sugar spheres | 78.00 |
| Purified water | q.s. |

### Manufacturing Process:

1. Hydroxypropylmethyl cellulose is dissolved in purified water to form a binder solution.
2. Doxycycline is added to the binder solution of step 1 to form a drug-binder solution.
3. Sugar spheres are coated with the drug-binder solution of step 2 to obtain coated pellets.
4. The coated pellets of step 3 are dried, then filled into capsules.

### Example 3

| | Quantity |
|---|---|
| Ingredients | (% w/w) |
| Doxycycline hyclate equivalent to doxycycline 32 mg | 18.47 |
| Polyvinyl pyrrolidone | 3.04 |
| Microcrystalline cellulose | 77.50 |
| Purified water | q.s. |
| Magnesium stearate | 1.00 |

### Manufacturing Process:

1. Polyvinyl pyrrolidone is dissolved in purified water to form a binder solution.
2. Doxycycline and microcrystalline cellulose are mixed together to obtain a blend.
3. The blend of step 2 is granulated using the binder solution of step 1.
4. The granules of step 3 are lubricated with magnesium stearate.
5. The lubricated granules of step 4 are compressed into tablets.

### Example 4

| | Quantity |
|---|---|
| Ingredients | (% w/w) |
| Doxycycline hyclate equivalent to doxycycline 34 mg | 19.62 |
| Polyvinyl pyrrolidone | 2.38 |
| Microcrystalline cellulose | 77.00 |
| Purified water | q.s. |
| Magnesium stearate | 1.00 |

### Manufacturing Process:

1. Polyvinyl pyrrolidone is dissolved in purified water to form a binder solution.
2. Doxycycline and microcrystalline cellulose are mixed together to obtain a blend.
3. The blend of step 2 is granulated using the binder solution of step 1.
4. The granules of step 3 are lubricated with magnesium stearate.
5. The lubricated granules of step 4 are compressed into tablets.

### Example 5

| | Quantity |
|---|---|
| Ingredients | (% w/w) |
| Doxycycline hyclate equivalent to doxycycline 32 mg | 18.47 |
| Polyvinyl pyrrolidone | 3.04 |
| Microcrystalline cellulose | 77.50 |
| Purified water | q.s. |
| Magnesium stearate | 1.00 |

### Manufacturing Process:

1. Polyvinyl pyrrolidone is dissolved in purified water to form a binder solution.
2. Doxcycline is added to the binder solution of step 1 to form a drug-binder solution.
3. Microcrystalline cellulose is granulated using the drug-binder solution of step 2.
4. The granules of step 3 are lubricated with magnesium stearate.
5. The lubricated granules of step 4 are compressed into tablets.

### Example 6

| | Quantity |
|---|---|
| Ingredients | (% w/w) |
| Doxycycline hyclate equivalent to doxycycline 34 mg | 19.62 |
| Polyvinyl pyrrolidone | 2.38 |
| Microcrystalline cellulose | 77.00 |
| Purified water | q.s. |
| Magnesium stearate | 1.00 |

### Manufacturing Process:

1. Polyvinyl pyrrolidone is dissolved in purified water to form a binder solution.
2. Doxcycline is added to the binder solution of step 1 to form a drug-binder solution.
3. Microcrystalline cellulose is granulated using the drug-binder solution of step 2.
4. The granules of step 3 are lubricated with magnesium stearate.
5. The lubricated granules of step 4 are compressed into tablets.

### Example 7

| | Quantity |
|---|---|
| Ingredients | (% w/w) |
| Doxycycline hyclate equivalent to doxycycline 32 mg | 18.47 |
| Polyvinyl pyrrolidone | 3.54 |
| Microcrystalline cellulose | 77.00 |
| Magnesium stearate | 1.00 |

### Manufacturing Process:

1. Doxycycline, microcrystalline cellulose, and polyvinyl pyrrolidone are mixed together to obtain a blend.
2. The blend of step 1 is lubricated with magnesium stearate.
3. The lubricated blend of step 2 is compressed into tablets.

### Example 8

| | Quantity |
|---|---|
| Ingredients | (% w/w) |
| Doxycycline hyclate equivalent to doxycycline 34 mg | 19.62 |
| Polyvinyl pyrrolidone | 2.38 |
| Microcrystalline cellulose | 77.00 |
| Magnesium stearate | 1.00 |

### Manufacturing Process:

1. Doxycycline, microcrystalline cellulose, and polyvinyl pyrrolidone are mixed together to obtain a blend.
2. The blend of step 1 is lubricated with magnesium stearate.
3. The lubricated blend of step 2 is compressed into tablets.

### Example 9

| | Quantity |
|---|---|
| Ingredients | (% w/w) |
| Doxycycline monohydrate equivalent to doxycycline 32 mg | 17.69 |
| Hydroxypropylmethyl cellulose | 2.91 |
| Sugar spheres | 79.40 |
| Purified water | q.s. |

### Manufacturing Process:

1. Hydroxypropylmethyl cellulose is dissolved in purified water to obtain a binder solution.
2. Doxycycline is added to the binder solution of step 1 to form a drug-binder solution.
3. Sugar spheres are coated with the drug-binder solution of step 2 to obtain coated pellets.
4. The coated pellets of step 3 are dried, then filled into capsules.

### Example 10

| | Quantity |
|---|---|
| Ingredients | (% w/w) |
| Doxycycline monohydrate equivalent to doxycycline 34 mg | 17.69 |
| Polyvinyl pyrrolidone | 2.48 |
| Microcrystalline cellulose | 78.83 |
| Magnesium stearate | 1.00 |

### Manufacturing Process:

1. Doxycycline, microcrystalline cellulose, and polyvinyl pyrrolidone are mixed together to obtain a blend.
2. The blend of step 1 is lubricated with magnesium stearate.
3. The lubricated blend of step 2 is compressed into tablets.

### Example 11

| | Quantity |
|---|---|
| Ingredients | (% w/w) |
| Doxycycline monohydrate equivalent to doxycycline 32 mg | 16.65 |
| Polyvinyl pyrrolidone | 3.86 |
| Microcrystalline cellulose | 78.50 |
| Purified water | q.s. |
| Magnesium stearate | 1.00 |

### Manufacturing Process:

1. Polyvinyl pyrrolidone is dissolved in purified water to form a binder solution.
2. Doxcycline is added to the binder solution of step 1 to form a drug-binder solution.
3. Microcrystalline cellulose is granulated using the drug-binder solution of step 2.
4. The granules of step 3 are lubricated with magnesium stearate.
5. The lubricated granules of step 4 are compressed into tablets.

### Example 12

| | Quantity |
|---|---|
| Ingredients | (% w/w) |
| Immediate Release Portion | |
| Doxycycline hyclate equivalent to doxycycline base 24 mg | 9.60 |
| Microcrystalline cellulose (Avicel ® PH 102) | 33.50 |
| Crospovidone | 2.91 |
| Polyvinyl pyrrolidone | 1.70 |
| Colloidal silicon dioxide | 0.24 |
| Iron oxide yellow | 0.49 |
| Magnesium stearate | 0.10 |
| Controlled Release Portion | |
| Doxycycline hyclate equivalent to doxycycline base 16 mg | 6.40 |
| Microcrystalline cellulose (Avicel ® PH 102) | 16.38 |
| Microcrystalline cellulose (Avicel ® PH 200) | 13.88 |
| Hydroxypropylmethyl cellulose (Methocel ® K4M Premium CR) | 4.85 |
| Hydroxypropylmethyl cellulose (Methocel ® K100 Premium LV) | 4.85 |
| Polyvinyl pyrrolidone | 1.46 |
| Colloidal silicon dioxide | 0.24 |
| Magnesium stearate | 0.49 |
| Coating | |
| Opadry ® | 2.91 |
| Purified water | q.s. |

Manufacturing process:
Immediate release portion
   1. Doxycycline hyclate, Avicel® PH 102, iron oxide yellow, crospovidone, polyvinyl pyrrolidone, colloidal silicon dioxide, and magnesium stearate were mixed to form a blend.
Controlled release portion
   2. Doxycycline hyclate, Avicel® PH 102, Avicel® PH 200, Methocel® K4M, Methocel® K100, polyvinyl pyrrolidone, and colloidal silicon dioxide were mixed to form a blend.
   3. The blend of step 2 was lubricated with magnesium stearate to form a final blend.
Compression
   4. The blend of step 1 was compressed, followed by compression of the final blend of step 3 to form bilayer tablets.
   5. Opadry® was dispersed in purified water to form a dispersion.
   6. The tablets of step 4 were coated with the Opadry® dispersion of step 5.

### Example 13

| | Quantity |
|---|---|
| Ingredients | (% w/w) |
| Doxycycline hyclate equivalent to doxycycline base 34 mg | 28.08 |
| Microcrystalline cellulose (Avicel ® PH 102) | 60.77 |
| Crospovidone | 6.00 |
| Polyvinyl pyrrolidone | 3.50 |
| Colloidal silicon dioxide | 0.50 |
| Iron oxide yellow | 0.20 |
| Magnesium stearate | 1.00 |

### Manufacturing Process

1. Doxycycline hyclate, Avicel® PH 102, iron oxide yellow, crospovidone, polyvinyl pyrrolidone, colloidal silicon dioxide, and magnesium stearate were mixed to form a blend.
2. The blend of step 1 was compressed into tablets.

### Example 14

| | Quantity |
|---|---|
| Ingredients | (% w/w) |
| Doxycycline hyclate equivalent to doxycycline base 32 mg | 12.81 |
| Microcrystalline cellulose (Avicel ® PH 102) | 62.89 |
| Microcrystalline cellulose (Avicel ® PH 200) | 13.87 |
| Crospovidone | 2.91 |
| Polyvinyl pyrrolidone | 3.16 |
| Colloidal silicon dioxide | 0.49 |
| Magnesium stearate | 0.97 |
| Opadry ® | 2.91 |
| Purified water | q.s. |

### Manufacturing Process

1. Doxycycline hyclate, crospovidone, polyvinyl pyrrolidone and microcrystalline cellulose were blended.
2. The blend of step 1 was lubricated with colloidal silicon dioxide and magnesium stearate.
3. The lubricated blend of step 2 was compressed into tablets.
4. The tablets of step 3 were coated with Opadry®.

### Example 15

| | Quantity |
|---|---|
| Ingredients | (% w/w) |
| Doxycycline hyclate equivalent to doxycycline base 32 mg | 12.81 |
| Microcrystalline cellulose (Avicel ® PH 102) | 53.18 |
| Microcrystalline cellulose (Avicel ® PH 200) | 13.87 |
| Crospovidone | 2.91 |
| Polyvinyl pyrrolidone | 3.16 |
| Hydroxypropylmethyl cellulose (Methocel ® K4 MCR) | 2.43 |
| Hydroxypropylmethyl cellulose (Methocel ® K100 | 7.28 |
| premium LVCR) | |
| Colloidal silicon dioxide | 0.49 |
| Magnesium stearate | 0.97 |
| Opadry ® | 2.91 |
| Purified water | q.s. |

### Manufacturing Process

1. Doxycycline hyclate, crospovidone, polyvinyl pyrrolidone, microcrystalline cellulose, and hydroxypropylmethyl cellulose were blended.
2. The blend of step 1 was lubricated with colloidal silicon dioxide and magnesium stearate.
3. The lubricated blend of step 2 was compressed into tablets.
4. The tablets of step 3 were coated with Opadry®.

### Example 16

| | Quantity |
|---|---|
| Ingredients | (% w/w) |
| Doxycycline hyclate equivalent to doxycycline base 36 mg | 14.41 |
| Microcrystalline cellulose (Avicel ® PH102) | 61.29 |
| Microcrystalline cellulose (Avicel ® PH 200) | 13.87 |
| Crospovidone | 2.91 |
| Polyvinyl pyrrolidone | 3.16 |
| Colloidal silicon dioxide | 0.49 |
| Magnesium stearate | 0.97 |
| Opadry ® | 2.91 |
| Purified water | q.s. |

### Manufacturing Process

1. Doxycycline hyclate, crospovidone, polyvinyl pyrrolidone, and microcrystalline cellulose were blended.
2. The blend of step 1 was lubricated with colloidal silicon dioxide and magnesium stearate.
3. The lubricated blend of step 2 was compressed into tablets.
4. The tablets of step 3 were coated with Opadry®.

### Example 17

| | Quantity |
|---|---|
| Ingredients | (% w/w) |
| Doxycycline hyclate equivalent to doxycycline base 36 mg | 14.41 |
| Microcrystalline cellulose (Avicel ® PH 102) | 51.58 |
| Microcrystalline cellulose (Avicel ® PH 200) | 13.87 |
| Crospovidone | 2.91 |
| Polyvinyl pyrrolidone | 3.16 |
| Hydroxypropylmethyl cellulose (Methocel ® K4 MCR) | 2.43 |
| Hydroxypropylmethyl cellulose (Methocel ® K100 | 7.28 |
| premium LVCR) | |
| Colloidal silicon dioxide | 0.49 |
| Magnesium stearate | 0.97 |
| Opadry ® | 2.91 |
| Purified water | q.s. |

### Manufacturing Process

1. Doxycycline hyclate, crospovidone, polyvinyl pyrrolidone, microcrystalline cellulose, and hydroxypropylmethyl cellulose were blended.
2. The blend of step 1 was lubricated with colloidal silicon dioxide and magnesium stearate.
3. The lubricated blend of step 2 was compressed into tablets.
4. The tablets of step 3 were coated with Opadry®.

### Example 18

| | Quantity |
|---|---|
| Ingredients | (% w/w) |
| Doxycycline hyclate equivalent to doxycycline base 36 mg | 14.41 |
| Microcrystalline cellulose (Avicel ® PH 102) | 46.72 |
| Microcrystalline cellulose (Avicel ® PH 200) | 13.87 |
| Crospovidone | 2.91 |
| Polyvinyl pyrrolidone | 3.16 |
| Hydroxypropylmethyl cellulose (Methocel ® K4 MCR) | 7.28 |
| Hydroxypropylmethyl cellulose (Methocel ® K100 | 7.28 |
| premium LVCR) | |
| Colloidal silicon dioxide | 0.49 |
| Magnesium stearate | 0.97 |
| Coating | |
| Opadry ® | 2.91 |
| Purified water | q.s. |

### Manufacturing Process

1. Doxycycline hyclate, crospovidone, microcrystalline cellulose, hydroxypropylmethyl cellulose, and polyvinyl pyrrolidone were blended.
2. The blend of step 1 was lubricated with colloidal silicon dioxide and magnesium stearate.
3. The lubricated blend of step 2 was compressed into tablets.
4. The tablets of step 3 were coated with Opadry®.

### Example 19

| | Quantity |
|---|---|
| Ingredients | (% w/w) |
| Doxycycline hyclate equivalent to doxycycline base 36 mg | 14.41 |
| Microcrystalline cellulose (Avicel ® PH 102) | 49.15 |
| Microcrystalline cellulose (Avicel ® PH 200) | 13.87 |
| Crospovidone | 2.91 |
| Polyvinyl pyrrolidone | 3.16 |
| Hydroxypropylmethyl cellulose (Methocel ® K100 | 12.14 |
| premium LVCR) | |
| Colloidal silicon dioxide | 0.49 |
| Magnesium stearate | 0.97 |
| Opadry ® | 2.91 |
| Purified water | q.s. |

### Manufacturing Process

1. Doxycycline hyclate, crospovidone, polyvinyl pyrrolidone, microcrystalline cellulose, and hydroxypropylmethyl cellulose were blended.
2. The blend of step 1 was lubricated with colloidal silicon dioxide and magnesium stearate.
3. The lubricated blend of step 2 was compressed into tablets.
4. The tablets of step 3 were coated with Opadry®.

### Example 20

| | Quantity |
|---|---|
| Ingredients | (% w/w) |
| Doxycycline hyclate equivalent to doxycycline base 36 mg | 14.41 |
| Microcrystalline cellulose (Avicel ® PH 102) | 43.95 |
| Microcrystalline cellulose (Avicel ® PH 200) | 13.86 |
| Tartaric acid pellets | 5.20 |
| Crospovidone | 2.91 |
| Polyvinyl pyrrolidone | 3.16 |
| Hydroxypropylmethyl cellulose (Methocel ® K100 | 12.14 |
| premium LVCR) | |
| Colloidal silicon dioxide | 0.49 |
| Magnesium stearate | 0.97 |
| Opadry ® | 2.91 |
| Purified water | q.s. |

### Manufacturing Process

1. Doxycycline hyclate, crospovidone, microcrystalline cellulose, hydroxypropylmethyl cellulose, polyvinyl pyrrolidone, and tartaric acid were blended.
2. The blend of step 1 was lubricated with colloidal silicon dioxide and magnesium stearate.
3. The lubricated blend of step 2 was compressed into tablets.
4. The tablets of step 3 were coated with Opadry®.

### Example 21

| | Quantity |
|---|---|
| Ingredients | (% w/w) |
| Doxycycline hyclate equivalent to doxycycline base 32 mg | 12.81 |
| Microcrystalline cellulose (Avicel ® PH 102) | 45.55 |
| Microcrystalline cellulose (Avicel ® PH 200) | 13.87 |
| Tartaric acid pellets | 5.20 |
| Crospovidone | 2.91 |
| Polyvinyl pyrrolidone | 3.16 |
| Hydroxypropylmethyl cellulose (Methocel ® K100 | 12.14 |
| premium LVCR) | |
| Colloidal silicon dioxide | 0.49 |
| Magnesium stearate | 0.97 |
| Opadry ® | 2.91 |
| Purified water | q.s. |

### Manufacturing Process

1. Doxycycline hyclate, crospovidone, microcrystalline cellulose, hydroxypropylmethyl cellulose, polyvinyl pyrrolidone, and tartaric acid were blended.
2. The blend of step 1 was lubricated with colloidal silicon dioxide and magnesium stearate.
3. The lubricated blend of step 2 was compressed into tablets.
4. The tablets of step 3 were coated with Opadry®.

### Pharmacokinetic Studies

Doxycycline is virtually completely absorbed after oral administration. The comparison of intravenous and oral doses of doxycycline indicated lower absorption for oral administration in the range of 73% to 77% (Agwuh et al. “Pharmacokinetics and pharmacodynamics of the tetracyclines including glycylcyclines,” Journal of Antimicrobial Chemotherapy, 58(2):256-265(2006)). This may be due to site specific absorption in the upper part of gastrointestinal tract (GIT), i.e., in the duodenum region. The drug released beyond the duodenum remains unabsorbed.

The same is indicated from bioequivalence studies carried out on two formulations. Example 12 having an immediate release (IR) component as 60% had shown lower bioavailability w.r.t. the reference product Oracea® having an IR component as 75% as indicated from the results shown in Table 1.

TABLE 1
Comparative pharmacokinetic data from the studies
conducted under fasting conditions.

| | Example 12 | Oracea ® |
|---|---|---|
| IR:CR | 60:40 | 75:25 |
| | i.e. 24:16 mg | i.e. 30:10 mg |
| Cₘₐₓ (A/C) | 85.05 (75.87-95.33) | |
| AUC₀₋ₜ (A/C) | 76.73 (68.03-86.54) | |

As doxycycline is absorbed from the upper part of the GIT, low bioavailability is observed in controlled release (CR) formulation which may be attributed to unabsorbed portion from the CR component.

The Cₘₐₓ ratio, in fact, may also be empirically derived from the calculation of the ratio of IR contribution from both formulations (i.e., 60/75=0.8).

Thus, the IR component results in complete release and absorption of the drug.

Based on the above understanding, pharmacokinetic values were predicted for dosage forms containing 24 mg to 36 mg of only IR components. The bioequivalence criteria was calculated using software Phoenix 64 (WinNonlin 6.4). The results are shown in Table 2:

TABLE 2
Extrapolation of in vivo data carried out for Doxycycline Hyclate IR
formulations (24-36 mg) w.r.t. RLD Oracea ® 40 mg.

| | Pharmacokinetic Parameter | |
|---|---|---|
| | Cₘₐₓ | AUC |
| Formulation | T/R ratio (90% CI) | T/R ratio (90% CI) |
| 24 mg | 84.68 (76.73-93.46) | 76.29 (68.40-85.09) |
| 26 mg | 91.74 (83.12-101.25) | 82.65 (74.10-92.18) |
| 28 mg | 98.79 (89.51-109.04) | 89.00 (79.80-99.27) |
| 30 mg | 105.85 (95.91-116.83) | 95.36 (85.40-106.36) |
| 32 mg | 112.91 (102.30-124.61) | 101.72 (91.20-113.46) |
| 34 mg | 119.96 (108.70-132.40) | 108.08 (96.90-120.55) |
| 36 mg | 127.02 (115.09-140.19) | 114.43 (102.60-127.64) |

(Data used is a bioequivalence study done on the Example 1 formulation and reference product Oracea ® (40 mg) in healthy male volunteers (N = 35) in a fasting state)

The data shows that formulations containing 24-36 mg of doxycycline would be similar to existing RLD (Oracea®) in terms of pharmacokinetic parameters of Cₘₐₓ and/or AUC.

1. A low dose tablet or capsule comprising a pharmaceutical composition that comprises 24 mg to 34 mg of doxycycline and one or more pharmaceutically acceptable excipients.

2. The tablet or capsule according to claim 1, wherein the pharmaceutical composition is intended for once daily administration.

3. The tablet or capsule according to claim 1, wherein the pharmaceutical composition comprises 30 mg to 34 mg of doxycycline.

4. The tablet or capsule according to claim 1, wherein the pharmaceutical composition comprises 32 mg to 34 mg of doxycycline.

5. The tablet or capsule according to claim 1, wherein the pharmaceutical composition comprises 24 mg of doxycycline.

6. The tablet or capsule according to claim 1, wherein the pharmaceutical composition comprises 26 mg of doxycycline.

7. The tablet or capsule according to claim 1, wherein the pharmaceutical composition comprises 28 mg of doxycycline.

8. The tablet or capsule according to claim 1, wherein the pharmaceutical composition comprises 30 mg of doxycycline.

9. The tablet or capsule according to claim 1, wherein the pharmaceutical composition comprises 32 mg of doxycycline.

10. The tablet or capsule according to claim 1, wherein the pharmaceutical composition comprises 34 mg of doxycycline.

11. (canceled)

12. The tablet or capsule according to claim 1, wherein the pharmaceutical composition is an immediate release composition.

13. (canceled)

14. A tablet or capsule comprising an immediate-release pharmaceutical composition that comprises 36 mg of doxycycline.
